(19) United States
(12) Patent Application Publication
(10) Pub. No.: US 2011/0200529 A1
(43) Pub. Date: Aug. 18, 2011

(54) ACTIVATABLE BIOLUMINESCENT PROBE SYSTEM AND METHOD OF USE THEREOF

(75) Inventors: Jianghong Rao, Sunnyvale, CA (US); Zuyong Xia, Palo Alto, CA (US)

(73) Assignee: STANFORD UNIVERSITY, Palo Alto, CA (US)

(21) Appl. No.: 13/119,721

(22) PCT Filed: Sep. 17, 2009
(86) PCT No.: PCT/US09/57368

§ 371 Date: Mar. 17, 2011

Related U.S. Application Data
(60) Provisional application No. 61/097,563, filed on Sep. 17, 2008.

(51) Int. Cl. A61K 49/00; G01N 21/76; C12M 1/34

(52) U.S. Cl. 424/9.1; 436/172; 422/82.08; 435/288.7

(57) ABSTRACT

A method of use of an activatable bioluminescent probe system includes: providing a bioluminescent protein and a quencher in a reaction environment; modifying a ligand between the quencher and the bioluminescent protein using a ligand interacting molecule; adding a bioluminescence initiating molecule to the reaction environment; and measuring light originating from the interaction between the bioluminescent protein and the bioluminescence initiating molecule.

### CROSS-REFERENCE TO RELATED APPLICATION(S)

This application claims the benefit of U.S. Provisional Patent Application Ser. No. 61/097,563 filed Sep. 17, 2008, and the subject matter thereof is incorporated herein by reference thereto.

### GOVERNMENT RIGHTS

This invention was made with Government support under contract W81XWH-06-1-0642 awarded by the Department of the Army. The Government has certain rights in this invention.

### TECHNICAL FIELD

The present invention relates generally to biochemical assay systems, and more particularly to an assay system incorporating an activatable bioluminescent probe for use both in vitro and in vivo.

### BACKGROUND ART

Recent advances in bioanalytical sciences and bioengineering have led to the development of bioluminescent reporter technology. Bioluminescence is the generation of light by a living organism. The components responsible for producing bioluminescence such as luciferases and luciferins have been isolated, characterized, and applied to the study of biology and disease. As a general bio-assaying technique, bioluminescence offers a powerful measurement method that is particularly useful in complex environments prone to autofluorescence.

Bioluminescent probes are customarily used to measure Adenosine Triphosphate (ATP). They are also used as reporter genes that express bioluminescent enzymes or proteins such as luciferases. Light emitted by the expressed luciferase is indicative that other gene manipulations in a plasmid or vector are also being expressed.

There is an important need to expand the functionality of bioluminescent probes to sense an expanding list of important biomarkers for biological processes and disease. These probes will be particularly useful for sensing biomarkers in complex environments such as biological fluids, tissues, and living organisms. Any bioluminescent probe assay that requires no separation of components following a reaction is particularly useful since separation steps can complicate the measurement. Assays not requiring separation steps are termed “homogeneous assays.”

Thus, a need still remains for bioluminescent probes with expanded functional characteristics for measuring biomarkers. In view of the expanding needs to understand biological processes and disease, it is increasingly critical that answers be found to these problems. In view of the ever-increasing commercial competitive pressures, along with growing consumer expectations and the diminishing opportunities for meaningful product differentiation in the marketplace, it is critical that answers be found for these problems. Additionally, the need to reduce costs, improve efficiencies and performance, and meet competitive pressures adds an even greater urgency to the critical necessity for finding answers to these problems.

Solutions to these problems have been long sought but prior developments have not taught or suggested any solutions and, thus, solutions to these problems have long eluded those skilled in the art.

### DISCLOSURE OF THE INVENTION

The present invention provides a method of use of an activatable bioluminescent probe system including: providing a bioluminescent protein and a quencher in a reaction environment; modifying a ligand between the quencher and the bioluminescent protein using a ligand interacting molecule; adding a bioluminescence initiating molecule to the reaction environment; and measuring light originating from the interaction between the bioluminescent protein and the bioluminescence initiating molecule.

In addition, the present invention provides an activatable bioluminescent probe system, including: a bioluminescent protein and a quencher in a reaction environment; a ligand interacting molecule for modifying a ligand between the quencher and the bioluminescent protein; a bioluminescence initiating molecule; and a detector for measuring the generation of light from the interaction between the bioluminescent protein and the bioluminescence initiating molecule.

Certain embodiments of the invention have other steps or elements in addition to or in place of those mentioned above. The steps or element will become apparent to those skilled in the art from a reading of the following detailed description when taken with reference to the accompanying drawings.

### BRIEF DESCRIPTION OF THE DRAWINGS

FIG. 1 is an activatable bioluminescent probe system in an embodiment of the present invention.

FIG. 2 is an activatable bioluminescent probe system for measuring the activity of a proteolytic enzyme.

FIG. 3 is an activatable bioluminescent probe system for measuring the presence of a ligand interacting molecule that mediates the formation of the ligand.

FIG. 4 is an embodiment of the activatable bioluminescent probe system for measuring the presence of a ligand interacting molecule of FIG. 3 that mediates the formation of the ligand.

FIG. 5 is an embodiment of the activatable bioluminescent probe system for measuring the presence of a ligand interacting molecule of FIG. 3 that mediates the formation of the ligand.

FIG. 6 is an embodiment of the activatable bioluminescent probe system for measuring the presence of a ligand interacting molecule using an extendable molecule.

FIG. 7 is an embodiment of the activatable bioluminescent probe system for measuring the presence of a target nucleic acid.

FIG. 8 is bioluminescence spectra showing quenching of the activatable bioluminescent probe system of FIG. 2.

FIG. 9 is emission spectra from the activatable bioluminescent probe system of FIG. 2 following interrogation with a bioluminescence initiating molecule shown in FIG. 2.

FIG. 10 is an activatable bioluminescent probe system for measuring the activity of a proteolytic enzyme.

FIG. 11 is emission spectra from the activatable bioluminescent probe system of FIG. 10 following interrogation with a bioluminescence initiating molecule.

FIG. 12 are sketches of molecular imaging of tumor sites in a host using the activatable bioluminescent probe system of FIG. 10.

FIG. 13 is a flow chart of a method of use of the activatable bioluminescent probe system in a further embodiment of the present invention.

### BEST MODE FOR CARRYING OUT THE INVENTION

The following embodiments are described in sufficient detail to enable those skilled in the art to make and use the invention. It is to be understood that other embodiments would be evident based on the present disclosure, and that system, process, or mechanical changes may be made without departing from the scope of the present invention.

In the following description, numerous specific details are given to provide a thorough understanding of the invention. However, it will be apparent that the invention may be practiced without these specific details. In order to avoid obscuring the present invention, some well-known circuits, system configurations, and process steps are not disclosed in detail.

The drawings showing embodiments of the system are semi-diagrammatic and not to scale and, particularly, some of the dimensions are for the clarity of presentation and are shown exaggerated in the drawing FIGs. Similarly, although the views in the drawings for ease of description generally show similar orientations, this depiction in the FIGs. is arbitrary for the most part. Generally, the invention can be operated in any orientation.

The same numbers are used in all the drawing FIGs. to relate to the same elements. The embodiments have been numbered first embodiment, second embodiment, etc. as a matter of descriptive convenience and are not intended to have any other significance or provide limitations for the present invention.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

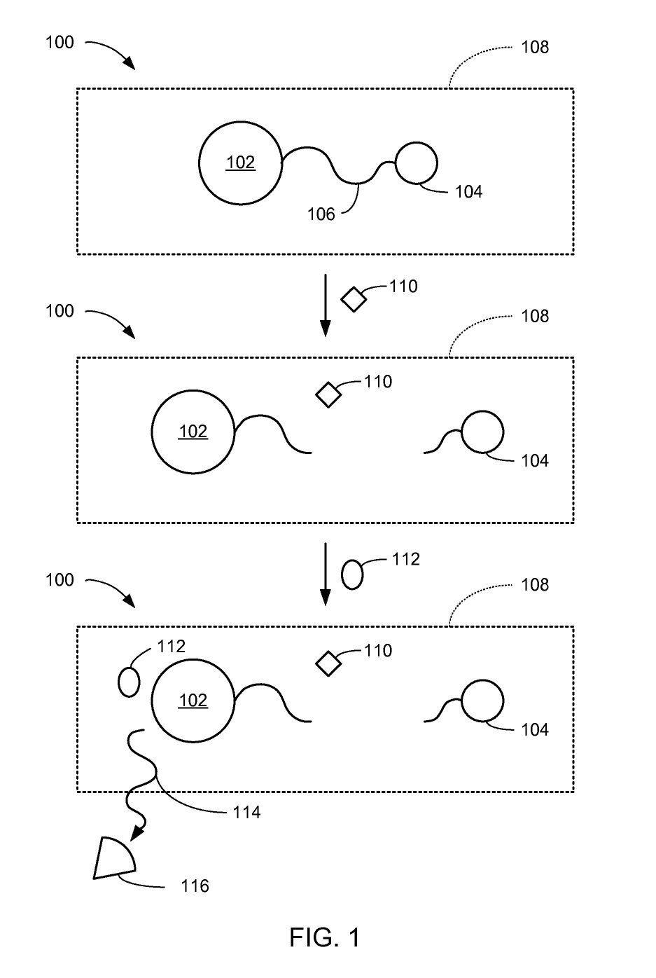

Referring now to FIG. 1, therein is shown an activatable bioluminescent probe system 100 in an embodiment of the present invention. The activatable bioluminescent probe system 100 includes a bioluminescent protein 102, a ligand 104, and a quencher 106. Activation of the activatable bioluminescent probe system 100 occurs when the quencher 104 is separated from the bioluminescent protein 102. The activatable bioluminescent probe system 100 is generally implemented within a reaction environment 108.

The term “bioluminescent protein” as used herein refers to a protein capable of participating in a light generating reaction involving a bioluminescence initiator molecule. Bioluminescence is the emission of light by biological molecules, particularly proteins. Luciferases and luciferase photoproteins are examples of the bioluminescent protein 102.

A luciferase is an enzyme that catalyzes a light emitting reaction. Examples of luciferases include firefly, Renilla, Gaussia, and Pleuroramma luciferases. Luciferases are proteins that occur naturally in an organism and can also be produced using recombinant engineering techniques with engineered amino acid sequences and three dimensional conformations.

The term “bioluminescence initiator molecule” as used herein refers to a molecule that interacts with the bioluminescent protein 102 to generate bioluminescence. The bioluminescence initiator molecule includes, but is not limited to luciferins and coelenterazine, analogs thereof, and functional derivatives thereof Derivatives of coelenterazine include, but are not limited to, coelenterazine 400a, coelenterazine cp, coelenterazine f, coelenterazine fcp, coelenterazine h, coelenterazine hcp; coelenterazine ip, coelenterazine n, coelenterazine O, coelenterazine c, coelenterazine c, coelenterazine i, coelenterazine icp, coelenterazine 2-methyl, benzyl-coelenterazine bisdeoxycoelenterazine, and deep blue coelenterazine (DBC) (described in more detail in U.S. Pat. Nos. 6,020,192; 5,968,750 and 5,874,304).

Luciferase photoproteins, such as aequorein and obelin, are apoproteins bound to a bioluminescence initiator molecule. A bioluminescent reaction involving the bioluminescent initiator molecule is generated when an ion such as calcium ion (Ca²⁺) triggers a conformational change in the photoprotein.

The term “quencher” as used herein refers to any organic or inorganic molecule capable of absorbing the light emitted by the reaction initiated or catalyzed by the bioluminescent protein 102. The quencher 104 is generally chosen such as its absorption spectrum partially or substantially overlaps the emission spectrum of the bioluminescent reaction associated with the bioluminescent protein 102. The quencher 104 may be any quenching dye or colloidal particle with an absorption spectrum partially or completely overlapping the emission spectrum of the bioluminescent protein 102. Among other examples, the quencher may be Black Hole Quencher (BHQ), 4-(dimethylaminoazo)benzene-4-carboxylic acid (DABCYL), Carboxytetramethylrhodamine (TAMRA), or colloidal gold.

The term “ligand” as used herein refers to any interaction or molecule that keeps the quencher 104 bound or in the proximity of the bioluminescent molecule 102. For example, the ligand 106 may be a non-covalent bond, a peptide linker, a polymer linker such as a carbon-chain linker, or a linker consisting of a nucleic acid chain or hybridized nucleic acid chains.

The term “reaction environment” as used herein is defined as any in vitro, in vivo, or ex vivo environment in which the activatable bioluminescent probe system 100 is implemented. The reaction environment may be a buffer solution; a biological liquid such as urine or blood, or a host among many other examples.

The term “host” as used herein refers to any living organism including mammals, humans, other living organisms, and samples such as cell or tissue taken from a living organism. A living organism can be as simple as a single eukaryotic cell or as complex as a human.

The ligand 106 may be modified by changes in the conditions of the reaction environment 108 or by interactions with a ligand interacting molecule 110. For example, the ligand 106 may be disrupted by changes in ionic strength, temperature, or pH of the reaction environment 108, effectively terminating or weakening the ligand 106. For example, a change in temperature or pH of the reaction environment 108 may create “melting” when the ligand 106 is composed of hybridized nucleic acids, effectively terminating the ligand 106 thus separating the quencher 104 from the bioluminescent protein 102.

The ligand interacting molecule 110 may create or catalyze formation, cleavage, or a conformational change of the ligand 106. Thus, in such embodiment of the present invention, the activatable bioluminescent probe system 100 becomes a sensor for the presence of the ligand interacting molecule 110. An example of a change in conformation of the ligand 106 includes the extension of a coiled structure into a longer structure, extending the ligand 106 sufficiently to reduce the effect of the quencher 104 on the bioluminescent enzyme 102.

Interrogation of the activation state of the activatable bioluminescent probe system 100 may be performed by adding a bioluminescence initiating molecule 112 to the reaction environment 108, initiating the generation of light 114 which is indicative of the action of the quencher 104 on the bioluminescent protein 102. The light 114 generated by the interrogation of the activatable bioluminescent probe system 100 is measured using a detector 116 such as a single-point detector or an image sensor. Examples of the detector 116 include a photodiode, an avalanche photodiode, a Geiger-Mode photodiode, a photomultiplier tube, a CMOS camera, a CCD camera, a cooled CCD camera, an intensified CCD camera, or an electron multiplying CCD camera among several other possibilities.

It has been unexpectedly discovered that positioning the quencher 104 in the proximity of the bioluminescent protein 102 through the ligand 106 can effectively reduce or quench bioluminescence emission generated by the interaction between the bioluminescent protein 102 and the bioluminescence initiating molecule 112. It has also been unexpectedly found that the quenching of the bioluminescent protein 102 may be used as a sensor of changes in the reaction environment 108 or a sensor for the presence of the ligand interacting molecule 110.

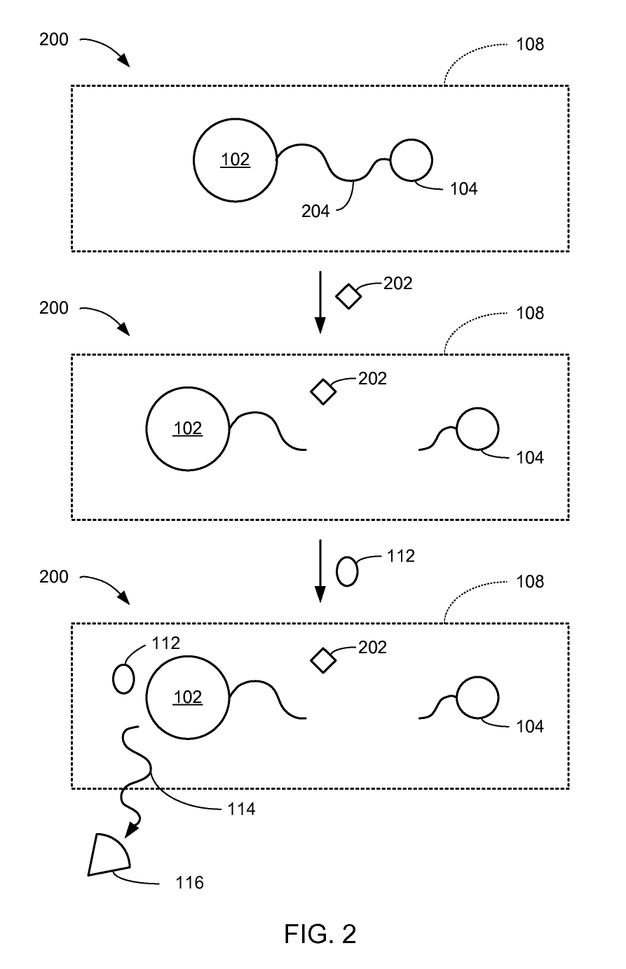

Referring now to FIG. 2, therein is shown an activatable bioluminescent probe system 200 for measuring the activity of a proteolytic enzyme 202. In this embodiment of the present invention, a peptide linker 204 that includes a peptide substrate specific to the proteolytic enzyme 202 is the ligand 106 shown in FIG. 1. The peptide linker 204 may include other linking molecules in addition to the peptide substrate. Cleavage of the peptide linker 204 catalyzed by the proteolytic enzyme 202 releases the quencher 104 from the proximity of the bioluminescent protein 202, activating the activatable bioluminescent probe system 200.

The activity of the proteolytic enzyme 202 is thus measured by sensing the light 114 originating from the interaction between the bioluminescent protein 102 and the bioluminescence initiating molecule 112. For example, exposure to the proteolytic enzyme 202 results in the cleavage of the peptide linker 204, separating the quencher 104 from the bioluminescent enzyme 102. Subsequent exposure to the bioluminescence initiating molecule 112 results in the generation of the light 114 in correlation to the number of cleavage sites, and thus the activity of the proteolytic enzyme 202.

The terms “proteolytic enzyme,” “protease,” and “peptidase” as interchangeably used herein refer to an enzyme that conducts proteolysis, that is, cleaves a peptide or a protein by hydrolysis of a peptide bond that links one amino acid to another in the peptide chain. Proteases occur naturally in all organisms.

Proteolytic enzymes are involved in a multitude of physiological reactions from simple digestion of food proteins to highly-regulated cascades (e.g., the blood-clotting cascade, the complement system, apoptosis pathways). Proteases can be classified into six groups: serine proteases; threonine proteases; cysteine proteases; aspartic acid proteases; metalloproteases; and glutamic acid proteases. Assignment of a protease to a certain group depends on the structure of catalytic site and the amino acid (as one of the constituents) essential for its activity.

The mechanism used to cleave a peptide bond involves making an amino acid residue that has the cysteine and threonine (peptidases), or a water molecule (aspartic acid, metallo- and glutamic acid peptidases) nucleophilic so that it can attack the peptide carbonyl group.

Some proteases can detach the terminal amino acids from a peptide or a protein (exopeptidases); the others attack internal peptide bonds of a protein (endopeptidases, such as trypsin, chymotrypsin, pepsin, papain, elastase). Thus, some peptidases can recognize specific sequences within the amino acid sequence of a peptide, and then cleave a specific peptide bond between two defined adjacent amino acid residues. Alternatively a protease can break down a complete peptide to individual amino acids. The activity can be destructive, completely abolishing a protein's function, or a partial digestion at one or more specific sites within the polypeptide sequence that may lead to the inactivation of a protein activity, or to the release of a cleaved product that is either activated or has a novel activity or function.

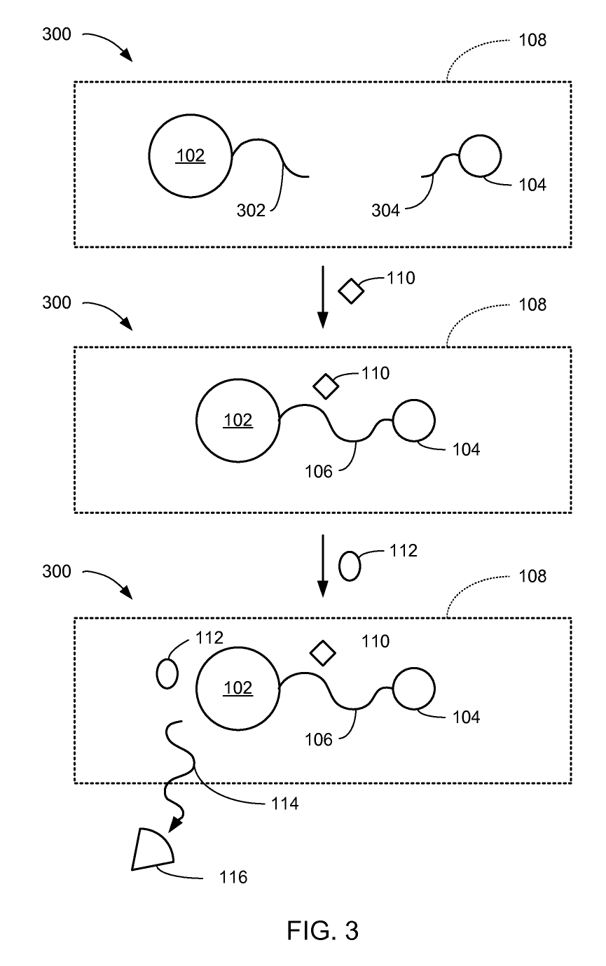

Referring now to FIG. 3, therein is shown an activatable bioluminescent probe system 300 for measuring the presence of a ligand interacting molecule 110 that mediates the formation of the ligand 106. In this embodiment of the present invention, the bioluminescent protein 102 is attached to a first ligand fragment 302 and the quencher 104 is attached to a second ligand fragment 304. The presence of the ligand interacting molecule 110 triggers the formation of the ligand 106, de-activating the activatable bioluminescent probe system 300.

The presence of the ligand interacting molecule 110 may be quantified by measuring the light 114 originating from the interaction between the bioluminescent protein 102 and the bioluminescence initiating molecule 112 using the detector 116 once the system is interrogated by adding the bioluminescence initiating molecule 112 to the reaction environment 108. The light 114 originating from the activatable bioluminescent probe system 300 is reduced with increased concentrations of the ligand interacting molecule 110.

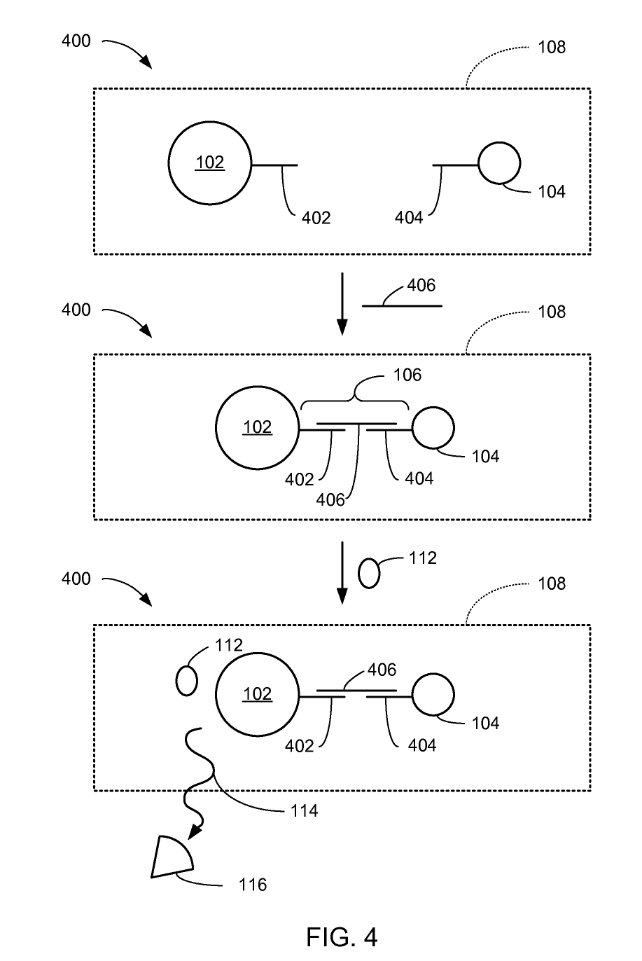

Referring now to FIG. 4, therein is shown an embodiment of the activatable bioluminescent probe system 400 for measuring the presence of a ligand interacting molecule 110 of FIG. 3 that mediates the formation of the ligand 106. In this embodiment of the present invention, a first nucleic acid probe 402 is the first ligand fragment 302 shown in FIG. 3, a second nucleic acid probe 404 is the second ligand fragment 304 of FIG. 3, and a target nucleic acid 406 is the ligand interacting molecule 110 shown in FIG. 3.

A portion of the first nucleic acid probe 402 furthest from the bioluminescent protein 102 contains a nucleotide sequence that is complementary to the nucleotide sequence of one end of the target nucleic acid 406, and a portion of the second nucleic acid probe 404 furthest from the quencher 104 contains a nucleotide sequence that is complementary to the nucleotide sequence of the other end of a target nucleic acid 406.

Presence of the target nucleic acid 406 under the appropriate conditions in the reaction environment 108 triggers a hybridization reaction of the target nucleic acid 406 to the first nucleic acid probe 402 and the second nucleic acid probe 404, completing the formation of the ligand 106. Thus the presence of the target nucleic acid 406 may be quantified by measuring the light 114 originating from the interaction between the bioluminescent protein 102 and the bioluminescence initiating molecule 112 once the system is interrogated by adding the bioluminescence initiating molecule 112 to the reaction environment 108. The light 114 originating from the activatable bioluminescent probe system 400 is reduced with increased concentrations of the target nucleic acid 406.

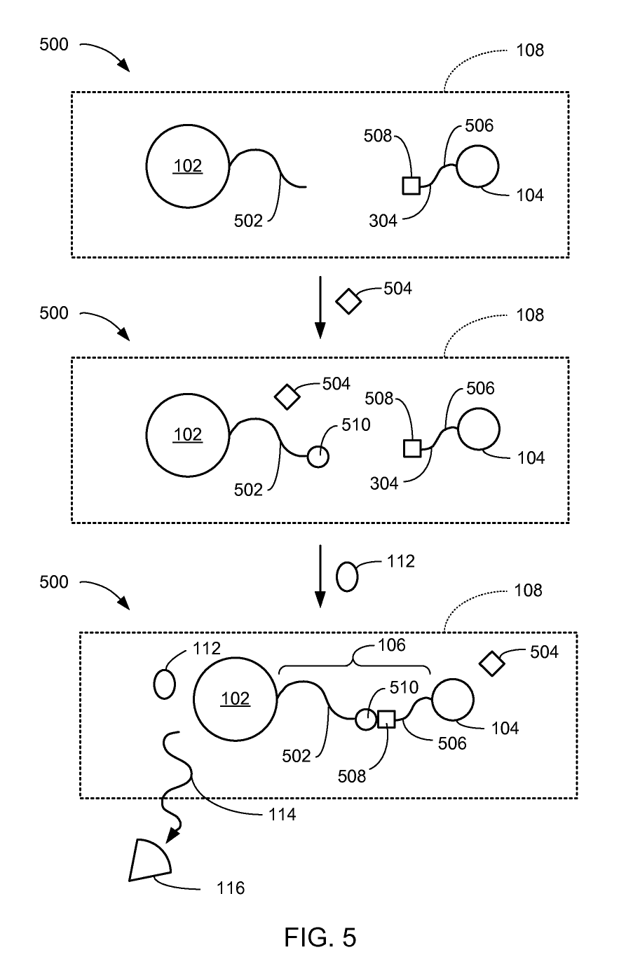

Referring now to FIG. 5, therein is shown an embodiment of the activatable bioluminescent probe system 500 for measuring the presence of a ligand interacting molecule 110 of FIG. 3 that mediates the formation of the ligand 106. In this embodiment of the present invention, a peptide linker 502 is the first ligand fragment 302 shown in FIG. 3 and a kinase enzyme 504 is the ligand interacting molecule 110 shown in FIG. 3. The second ligand fragment 304 includes a linker 506 and a phosphate-affinity molecule 508. The peptide linker 502 is a substrate specific to the kinase enzyme 504.

The term “kinase enzyme” as used herein refers to an enzyme that catalyses the phosphorylation of a molecule such as a peptide linker. Typically the phosphate group is transferred from high-energy donor molecules such as adenosine triphosphate (ATP). Presence of the kinase enzyme 504 under the appropriate conditions in the reaction environment 108 catalyzes the formation of a phosphate group 510 on the peptide linker 502, which subsequently becomes attached to the phosphate-affinity molecule 508 in the second ligand fragment 304, completing the formation of the ligand 106.

The activity of kinase enzyme 504 may be quantified by measuring the light 114 originating from the interaction between the bioluminescent protein 102 and the bioluminescence initiating molecule 112 using the detector 116 once the system is interrogated by adding the bioluminescence initiating molecule 112 to the reaction environment 108. The light 114 originating from the activatable bioluminescent probe system 500 is reduced with increased concentrations of the kinase enzyme 504.

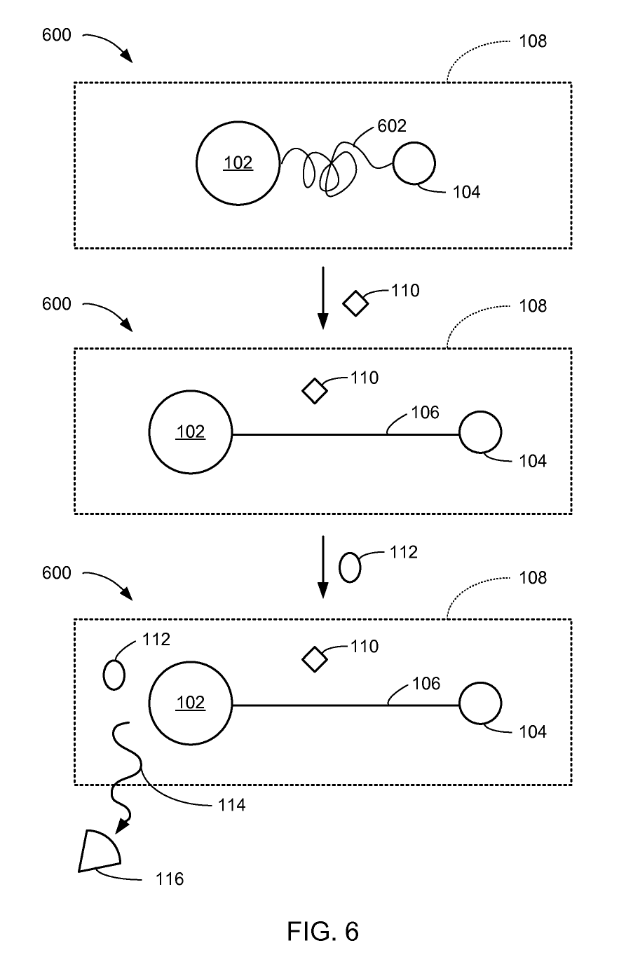

Referring now to FIG. 6, therein is shown an embodiment of the activatable bioluminescent probe system 600 for measuring the presence of a ligand interacting molecule 110 using an extendable molecule 602. In this embodiment of the present invention the extendable molecule 602 may be a linker in a compressed conformation such as a coiled or hairpin structure, and is one embodiment of the ligand 106 shown in FIG. 1. The ligand interacting molecule 110 attaches to the extendable molecule 602, extending it and increasing a distance between the bioluminescent protein 102 and the quencher 104.

The activatable bioluminescent probe system 600 is activated when the extendable molecule 602 is extended, and de-activated when the extendable molecule 602 is in its compressed conformation. Thus the presence of the ligand interacting molecule 110 may be quantified by measuring the light 114 originating from the interaction between the bioluminescent protein 102 and the bioluminescence initiating molecule 112 once the system is interrogated by adding the bioluminescence initiating molecule 112 to the reaction environment 108. The light 114 originating from the activatable bioluminescent probe system 600 is increased with increased concentrations of the ligand interacting molecule 110.

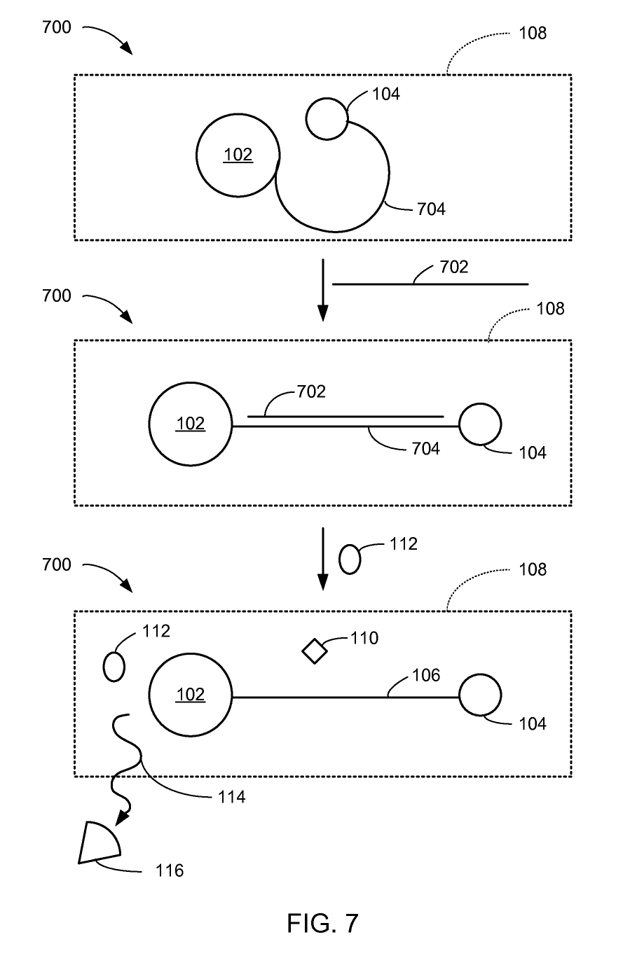

Referring now to FIG. 7, therein is shown an embodiment of the activatable bioluminescent probe system 700 for measuring the presence of a target nucleic acid 702. The target nucleic acid 702 may be a nucleic acid present in the reaction environment 108, added to the reaction environment 108 as part of a sample, or synthesized in-situ using the appropriate precursors. The target nucleic acid 702 is the ligand interacting molecule 110 shown in FIG. 6. A nucleic acid probe 704 serves as the ligand 106 of FIG. 6.

The nucleic acid probe 704 is a nucleic acid in compressed conformation that results in the placement of the quencher 104 in the proximity of the bioluminescent protein 102. The compressed conformation may be a coiled structure or a hairpin or loop structure among many other possibilities; for illustration purposes, a hairpin structure is shown in FIG. 7. Hybridization of the target nucleic acid 702 to the nucleic acid probe 704 that contains a complementary sequence to the target nucleic acid 702 results in the extension of the nucleic acid probe 704, activating the activatable bioluminescent probe system 700. Thus, in this embodiment of the present invention, the activatable bioluminescent probe system 700 is a sensor for the target nucleic acid 702.

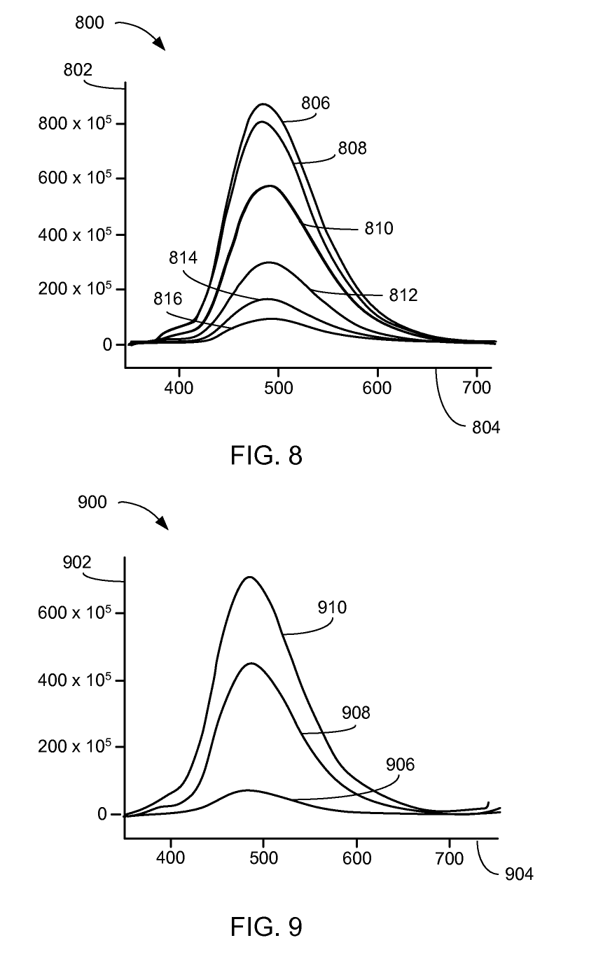

Referring now to FIG. 8, therein is shown bioluminescence spectra 800 showing quenching of the activatable bioluminescent probe system 200 of FIG. 2. It is noted that the bioluminescence spectra 800 is measured for a plurality of instances of the activatable bioluminescent probe system 200 of FIG. 2 in in vitro conditions.

The bioluminescence spectra 800 of FIG. 8 is recorded by exposing it to the bioluminescence initiating molecule 110 during the assembly of the structure incorporating the bioluminescent protein 102, the peptide linker 204, and the quencher 104 shown as the initial structure in FIG. 2. The assembly process for the initial state of the activatable bioluminescent probe system 200 results in quenching of the bioluminescent protein 102 of FIG. 2.

In this embodiment of the invention, the bioluminescent protein 102 of FIG. 2 is a luciferase, and more specifically, a mutated Renilla luciferase; the peptide linker 204 incorporates an amino acid sequence NH₂-Iso-Pro-Val-Ser-Leu-Arg-Ser-Gly-COOH, which is a substrate for the proteolytic enzyme known as matrix metallopeptidase-2; and the quencher 104 is a QXL 490 dye. Coelenterazine is the bioluminescence initiating molecule 110 used in the measurement of the emission spectrum 900.

The bioluminescence spectra 800 is recorded as relative luminescence units 802 as a function of emission wavelength 804.in units of nanometers. Bioluminescence protein spectrum 806 is the emission of the fluorescent protein 102 without being linked to the quencher 104. The first bioluminescence spectrum 808, second bioluminescence spectrum 810, third bioluminescence spectrum 812, fourth bioluminescence spectrum 814, fifth bioluminescence spectrum 816, and sixth bioluminescence spectrum 818 are emission spectra measured in ten minute intervals during the assembly of the activatable bioluminescent probe system 200.

The trend from the bioluminescence protein spectrum 806 to the sixth bioluminescence spectrum 818 shows quenching (de-activation) of the activatable bioluminescent probe system 200 during assembly.

Referring now to FIG. 9, therein is shown emission spectra 900 from the activatable bioluminescent probe system 200 of FIG. 2 following interrogation with a bioluminescence initiating molecule 110 shown in FIG. 2. It is noted that the bioluminescence spectra 900 is measured for a plurality of instances of the activatable bioluminescent probe system 200 of FIG. 2 in in vitro conditions.

In this embodiment of the present invention, the peptide linker 204 includes a peptide with the amino acid sequence NH₂-Iso-Pro-Val-Ser-Leu-Arg-Ser-Gly-COOH, which is specific to the proteolytic enzyme 202 known as matrix metallopeptidase-2 (MMP-2). The bioluminescent protein 102 is a luciferase, and more specifically, a mutated Renilla luciferase known as “Luc8.” Coelenterazine is the bioluminescence initiating molecule 110 used in the measurement of the emission spectrum 900.

The bioluminescence spectra 900 is recorded as relative luminescence units 902 as a function of emission wavelength 904.in units of nanometers. Bioluminescence spectrum 906 is the bioluminescence emission from the activatable bioluminescent probe system 200 of FIG. 2 in its initial quenched state. Incubation of the activatable bioluminescent probe system 200 in the presence of the proteolytic enzyme 202 results in cleavage of the peptide linker 204, separating the quencher 104 from the bioluminescent protein 102 as shown in FIG. 2.

Bioluminescence spectrum 906, bioluminescence spectrum 908, and bioluminescence spectrum 910 are the result of increased activation of the activatable bioluminescent probe system 200 through exposure to the proteolytic enzyme 202 as shown in FIG. 2.

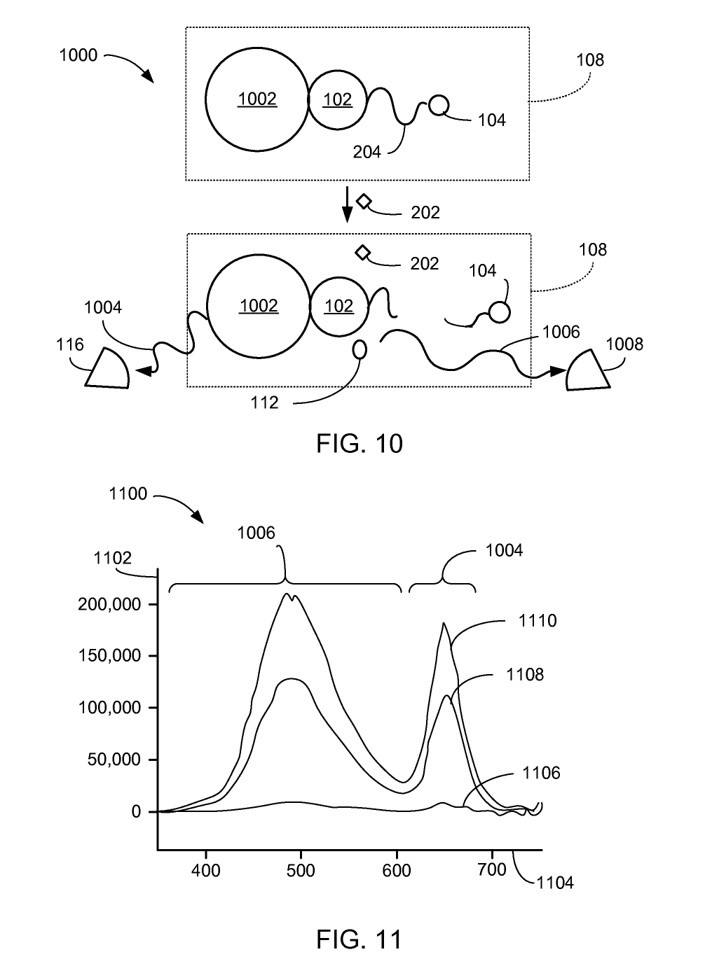

Referring now to FIG. 10, therein is shown an activatable bioluminescent probe system 1000 for measuring the activity of a proteolytic enzyme 202. In this embodiment of the present invention, a peptide linker 204 that includes a peptide substrate specific to the proteolytic enzyme 202 is the ligand 106 shown in FIG. 1. The peptide linker 204 may include other linking molecules in addition to the peptide substrate. Cleavage of the peptide linker 204 catalyzed by the proteolytic enzyme 202 releases the quencher 104 from the proximity of the bioluminescent protein 202, activating the activatable bioluminescent probe system 200.

In the activatable bioluminescent probe system 1000, the bioluminescence protein 102 is closely coupled to an energy acceptor 1002 such as a luminescent nanocrystal, a dye, or a fluorescent protein. When the bioluminescent protein 102 is exposed to the bioluminescence initiating compound 102 the energy that is generated by the combination of the bioluminescent protein 102 and the bioluminescence initiating compound 102 is directly transferred to the energy acceptor 1002 through a process known as Bioluminescence Resonance Energy Transfer (BRET). The BRET process generates luminescent emission 1004 from the energy acceptor 1002, typically at longer wavelengths than any bioluminescent protein emission 1006 from the bioluminescent protein 102.

The activity of the proteolytic enzyme 202 is thus measured by sensing the luminescent emission 1004 from the energy acceptor 1002 or the bioluminescent protein emission 1006 originating from the interaction between the bioluminescent protein 102 and the bioluminescence initiating molecule 112. The detector 116 is used to measure the luminescent emission 1004 using the appropriate light filters to distinguish it from the bioluminescent protein emission 1006. A second detector 1008 is employed to measure the bioluminescent protein emission 1006 also using the appropriate light filters.

In an alternate embodiment of the invention, the detector 116 may be used to measure the combination of the luminescent emission 1004 and the bioluminescent protein emission 1006.

Exposure to the proteolytic enzyme 202 results in the cleavage of the peptide linker 204, separating the quencher 104 from the bioluminescent enzyme 102. Subsequent exposure to the bioluminescence initiating molecule 112 results in the generation of the luminescent emission 1004 and the bioluminescent protein emission 1006 in correlation to the number of cleavage sites, and thus the activity of the proteolytic enzyme 202.

In the activatable bioluminescent probe system 1000, the luminescent nanocrystal 1002 may be chosen such that it emits the luminescent emission 1004 at wavelengths ranging from 600 to 950 nm to enable maximum imaging depth in a host. Operation in this wavelength range minimizes hemoglobin or water absorption. In addition, long wavelength operation reduces light scattering in tissues and autofluorescence from native proteins that may interfere with the measurement.

Alternate embodiments of the present invention include coupling of the energy acceptor 1002 to the bioluminescent protein 102 in all of the embodiments shown in FIG. 1 through FIG. 7, and FIG. 10. The coupling of the energy acceptor 1002 to the bioluminescent protein 102 generates a second source of light, the luminescent emission 1004.

Referring now to FIG. 11, therein is shown emission spectra 1100 from the activatable bioluminescent probe system 1000 of FIG. 10 following interrogation with a bioluminescence initiating molecule 110. It is noted that the bioluminescence spectra 1100 is measured for a plurality of instances of the activatable bioluminescent probe system 1000 of FIG. 10 in in vitro conditions.

In this embodiment of the present invention, the peptide linker 204 includes a peptide with the amino acid sequence NH₂-Iso-Pro-Val-Ser-Leu-Arg-Ser-Gly-COOH, which is specific to the proteolytic enzyme 202 known as matrix metallopeptidase-2 (MMP-2). The bioluminescent protein 102 is a luciferase, and more specifically, a mutated Renilla luciferase known as “Luc8.” Coelenterazine is the bioluminescence initiating molecule 110 used in the measurement of the emission spectrum 900.

The bioluminescence spectra 1100 is recorded as relative luminescence units 1102 as a function of emission wavelength 1104.in units of nanometers. The emission spectra 1100 from the activatable bioluminescent probe system 1000 of FIG. 10 includes the bioluminescent protein emission 1006 and the luminescent emission 1004.

Luminescent spectrum 1106 is the emission from the activatable bioluminescent probe system 1000 of FIG. 10 in its initial quenched state. Incubation of the activatable bioluminescent probe system 200 in the presence of the proteolytic enzyme 202 results in cleavage of the peptide linker 204, separating the quencher 104 from the bioluminescent protein 102 as shown in FIG. 10.

Bioluminescence spectrum 1108 and bioluminescence spectrum 1110 are the result of increased activation of the activatable bioluminescent probe system 1000 through exposure to the proteolytic enzyme 202 as shown in FIG. 10.

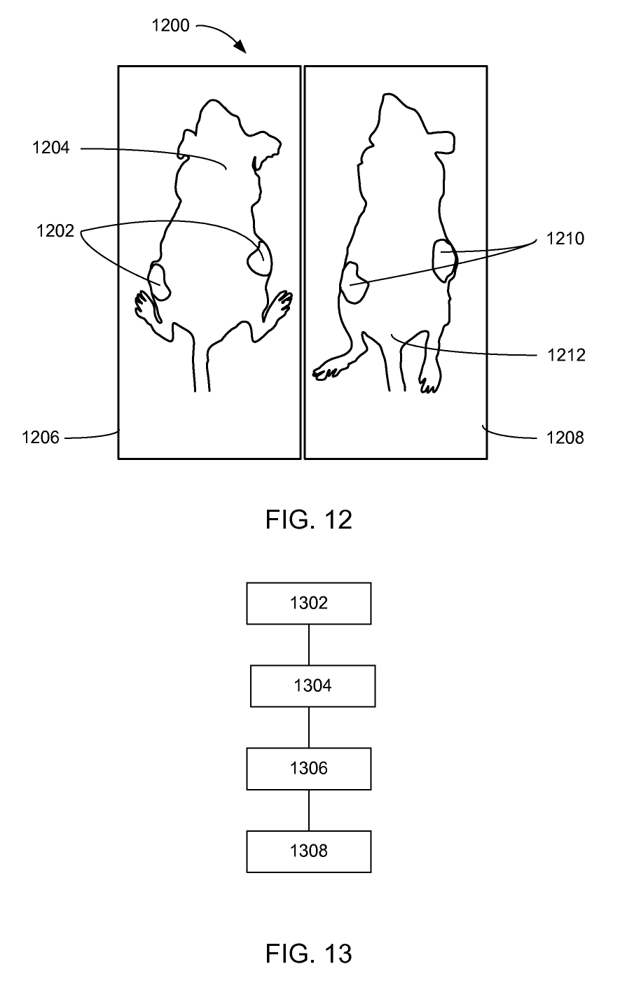

Referring now to FIG. 12, therein are shown sketches 1200 of molecular imaging of tumor sites 1202 in a host 1204 using the activatable bioluminescent probe system 1000 of FIG. 10. The host 1202 used in this example is a nude mouse and the tumor sites 1202 were grown using HT1080 tumor cells.

A first image trace 1206 shows the host 1204. The implanted tumor sites 1202 are clearly seen as bulging areas. A second image trace 1208 shows the bioluminescent protein emission 1006 and the luminescent emission 1004 measured 55 minutes after intravenous injection of 7 micrograms of the activatable bioluminescent probe system 1000 of FIG. 10. The bioluminescent protein emission 1006 and the luminescent emission 1004 are activated by the presence of the proteolytic enzyme 202 of FIG. 10 in the host.

As seen in the second image 1208, the implanted tumor site signals 1210 are distinguishable from the background signal. 1212 (in grayscale these regions are seen as lighter gray surrounded by darker gray).

Referring now to FIG. 13, therein is shown a flow chart of a method 1300 of use of the activatable bioluminescent probe system 100 in a further embodiment of the present invention. The method 1300 includes: providing a bioluminescent protein and a quencher in a reaction environment in a block 1302; modifying a ligand between the quencher and the bioluminescent protein using a ligand interacting molecule in a block 1304; adding a bioluminescence initiating molecule to the reaction environment in a block 1306; and measuring light originating from the interaction between the bioluminescent protein and the bioluminescence initiating molecule in a block 1308.

The resulting method, process, apparatus, device, product, and/or system is straightforward, cost-effective, uncomplicated, highly versatile, accurate, sensitive, and effective, and can be implemented by adapting known components for ready, efficient, and economical manufacturing, application, and utilization.

Another important aspect of the present invention is that it valuably supports and services the historical trend of reducing costs, simplifying systems, and increasing performance.

These and other valuable aspects of the present invention consequently further the state of the technology to at least the next level.

While the invention has been described in conjunction with a specific best mode, it is to be understood that many alternatives, modifications, and variations will be apparent to those skilled in the art in light of the aforegoing description. Accordingly, it is intended to embrace all such alternatives, modifications, and variations that fall within the scope of the included claims. All matters hithertofore set forth herein or shown in the accompanying drawings are to be interpreted in an illustrative and non-limiting sense.

1. A method of use of an activatable bioluminescent probe system comprising:
providing a bioluminescent protein and a quencher in a reaction environment;
modifying a ligand between the quencher and the bioluminescent protein using a ligand interacting molecule;
adding a bioluminescence initiating molecule to the reaction environment; and
measuring light originating from the interaction between the bioluminescent protein and the bioluminescence initiating molecule.

2. The method as claimed in claim 1 wherein modifying the ligand between the quencher and the bioluminescent protein using a ligand interacting molecule comprises cleaving the ligand.

3. The method as claimed in claim 1 wherein modifying the ligand between the quencher and the bioluminescent protein using a ligand interacting molecule comprises forming the ligand.

4. The method as claimed in claim 1 wherein modifying the ligand between the quencher and the bioluminescent protein using a ligand interacting molecule comprises extending the ligand.

5. The method as claimed in claim 1 wherein modifying the ligand is the result of changes in the reaction environment.

6. An activatable bioluminescent probe system comprising:
a bioluminescent protein and a quencher in a reaction environment;
a ligand interacting molecule for modifying a ligand between the quencher and the bioluminescent protein;
a bioluminescence initiating molecule; and
a detector for measuring the generation of light from the interaction between the bioluminescent protein and the bioluminescence initiating molecule.

7. The system as claimed in claim 6 wherein the ligand interacting molecule is a proteolytic enzyme.

8. The system as claimed in claim 6 wherein the ligand interacting molecule includes a target nucleic acid.

9. The system as claimed in claim 6 wherein the ligand interacting molecule is a kinase enzyme.

10. The system as claimed in claim 6 wherein the ligand interacting molecule is an extendable molecule.

11. A method of use of an activatable bioluminescent probe system comprising:
providing a bioluminescent protein linked to an energy acceptor, and a quencher in a reaction environment;
modifying a ligand between the quencher and the bioluminescent protein using a ligand interacting molecule;
adding a bioluminescence initiating molecule to the reaction environment; and
measuring luminescent emission initiated by the interaction between the bioluminescent protein and the bioluminescence initiating molecule.

12. The method as claimed in claim 11 wherein modifying the ligand between the quencher and the bioluminescent protein using a ligand interacting molecule comprises cleaving the ligand.

13. The method as claimed in claim 11 wherein modifying the ligand between the quencher and the bioluminescent protein using a ligand interacting molecule comprises forming the ligand.

14. The method as claimed in claim 11 wherein modifying the ligand between the quencher and the bioluminescent protein using a ligand interacting molecule comprises extending the ligand.

15. The method as claimed in claim 11 further comprising correlating the luminescent emission initiated by the interaction between the bioluminescent protein and the bioluminescence initiating molecule to properties of the ligand interacting molecule.

16. The system as claimed in claim 6 further comprising an energy acceptor linked to the bioluminescent protein and wherein the detector is for measuring luminescent emission.

17. The system as claimed in claim 6 further comprising a second detector for measuring the generation of light from the interaction between the bioluminescent protein and the bioluminescence initiating molecule.

18. The system as claimed in claim 6 wherein the reaction environment is a host.

19. The system as claimed in claim 6 wherein the detector is an image sensor.

20. The system as claimed in claim 6 wherein the second detector is an image sensor.
